(19) United States
(12) Patent Application Publication
(10) Pub. No.: US 2025/0200471 A1
(43) Pub. Date: Jun. 19, 2025

(54) SYSTEMS AND METHODS FOR DIGITAL DEVICE MIRRORING FOR HOLISTIC SUPPLY CHAIN MODELLING

(71) Applicant: JABIL INC., St. Petersburg, FL (US)

(72) Inventor: MILIND MORE, St. Petersburg, FL (US)

(21) Appl. No.: 18/848,549

(22) PCT Filed: Mar. 22, 2023
(86) PCT No.: PCT/US2023/015976

§ 371 Date: Sep. 19, 2024

Related U.S. Application Data
(60) Provisional application No. 63/322,619, filed on Mar. 22, 2022.

(51) Int. Cl. G06Q 10/0631

(52) U.S. Cl. CPC G06Q 10/06315

(57) ABSTRACT

Apparatus, system and method for providing a digital device health platform for optimizing an offering of a product. Disclosed embodiments include: a plurality of supply chains, each of which contributes a different aspect of the product; a plurality of input modules; a digital device health engine that receives processed data from each of the input modules, and that processes the received processed data to generate a holistic digital twin of all aspects of the product corresponding to the plurality of the inputs, which includes the supply chain data from each of the plurality of supply chains, wherein the corresponding comprises at least a correlating to an optimized level of performance of the purpose, a comparing to a plurality of rules governing the performance of the purpose, and feedback from machine learning regarding anonymized prior similar iterations of other products similar to the product as reflected by the third party data.

### CROSS-REFERENCE TO RELATED APPLICATIONS

This application is a U.S. national phase application filed under 35 U.S.C. § 371 claiming benefit to International Patent Application No. PCT/US23/15976, filed Mar. 22, 2023, which is entitled to priority of U.S. Provisional Application No. 63/322,619, filed Mar. 22, 2022, each of which is incorporated herein by reference as if set forth in its entirety.

### BACKGROUND

### Field of the Disclosure

The present disclosure relates to digitizing a holistic device profile. More specifically, the present disclosure is related to systems and methods for digital device mirroring for holistic supply chain development and modelling.

### Background of the Disclosure

In the presently known art, a “supply chain” exists for nearly all types of products and services, albeit a discreet supply chain for each discreet aspect of the product or service. That is, a supply chain exists for hardware devices, a separate supply chain exists for firmware development to operate the hardware of the device, a separate supply chain exists to develop and deploy algorithmic applications to operate the firmware and hardware of the device, and a separate supply chain exists to generate, manipulate, and deploy the data developed by the hardware, firmware, and software.

However, the discretization of the various supply chain aspects at present for a single product or service is highly inefficient. That is, cross disciplinary expertise are of little use in the current discretization of supply chains, and hence there is a knowledge loss as each discrete supply chain aspect is developed. Consequently, various problems typically arise during a product or service offering, in large measure because different aspects of the product or service have been developed, created, and/or manufactured by disconnected entities and supply chains that do not appreciate the problems that will be presented by other product or service aspects once all discrete supply chain aspects are combined into the final product or service offering.

The foregoing may particularly be the case in instances where the hardware device is operated in a high-stress or high-impact environment, such as in the medical field, where seamless operation between hardware, firmware, software, generated data, and feedback regarding all of the foregoing is a prerequisite to providing good outcomes for patients. Moreover, the data generated in, for example, the medical field pertains not only to devices but more particularly to patients, meaning the generated and manipulated data is not only vital to good health outcomes, but may additionally be subjected to a large variety of rules, laws, and confidentiality, such as with regard to both caregivers and patients, as well as regarding the relationship therebetween.

### SUMMARY OF THE DISCLOSURE

Disclosed is an apparatus, system and method for providing a digital device health platform for optimizing an offering of a product. The disclosed embodiments include: a plurality of supply chains, each of which contributes a different aspect of the product; and a plurality of input modules comprising: at least one third party data input for receiving third party data; a supply chain management input for receiving supply chain data from each of the plurality of supply chains; an application data input for receiving data regarding operation of at least one application operating in conjunction with hardware of the product to provide a purpose of the product; a device hardware data input for receiving data regarding operation of the hardware; a software application input for receiving purpose data from the at least one application; and a firmware input for receiving data regarding operation of firmware drivers for the hardware.

The platform further includes: a digital device health engine that receives processed data from each of the input modules, and that processes the received processed data, via at least one computer processor processing non-transitory computing code, to generate a holistic digital twin of all aspects of the product corresponding to the plurality of the inputs, which includes the supply chain data from each of the plurality of supply chains, wherein the corresponding comprises at least a correlating to an optimized level of performance of the purpose, a comparing to a plurality of rules governing the performance of the purpose, and feedback from machine learning regarding anonymized prior similar iterations of other products similar to the product as reflected by the third party data; and a plurality of outputs reflecting the corresponding to a graphical user interface.

### BRIEF DESCRIPTION OF THE DRAWINGS

The disclosure is illustrated by way of example and not limitation in the figures of the accompanying drawings, in which like references may indicate similar elements and in which:

FIG. 1 illustrates a supply chain that includes the transmitting and processing data, and particularly supply chain management (SCM) data, under an exemplary embodiment;

FIG. 2 illustrates an exemplary processing device suitable for use in the embodiment of FIG. 1 for processing and presenting SCM data;

FIG. 3 illustrates aspects of the embodiments;

FIG. 4 illustrates aspects of the embodiments;

FIG. 5 illustrates aspects of the embodiments;

FIG. 6 illustrates aspects of the embodiments; and

FIG. 7 illustrates aspects of the embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. Because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

Computer-implemented platforms, engines, systems and methods of use are disclosed herein that provide networked access to a plurality of types of digital content, including but not limited to video, image, text, audio, metadata, algorithms, interactive and document content, and that track, deliver, manipulate, transform and report the accessed content. Described embodiments of these platforms, engines, systems and methods are intended to be exemplary and not limiting. As such, it is contemplated that the herein described systems and methods may be adapted to provide many types of server and cloud-based manipulations, interactions, data exchanges, and the like, and may be extended to provide enhancements and/or additions to the exemplary platforms, engines, systems and methods described. The disclosure is thus intended to include all such extensions.

Furthermore, it will be understood that the terms “module” or “engine”, as used herein does not limit the functionality to particular literal modules, but may include any number of tangibly-embodied software and/or hardware components having a transformative effect on at least a portion of a system, and more particularly of the data input thereto and output therefrom. In general, a computer program product in accordance with the embodiments may comprise a tangible computer usable medium (e.g., standard RAM, an optical disc, a USB drive, or the like) having computer-readable program code embodied therein, wherein the computer-readable program code is adapted to be executed by at least one processor (working in connection with an operating system) to implement one or more functions and methods as described below. In this regard, the program code may be implemented in any desired language, and may be implemented as machine code, assembly code, byte code, interpretable source code or the like (e.g., via C, C++, C#, Java, Actionscript, Objective-C, Javascript, CSS, XML, etc.).

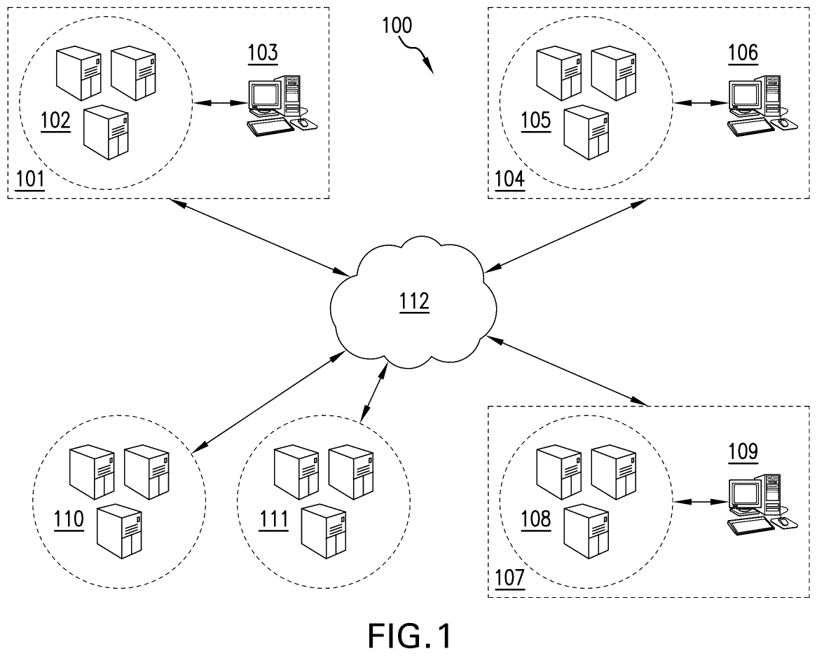

Turning to FIG. 1, an exemplary system is disclosed in an embodiment. In this example, system 100 is configured as a plurality of supply chain nodes in a typical supply chain, i.e., wherein each supply chain node is part of one distinct supply chain aspect, i.e., the hardware or the software or the firmware or the data management. The primary central hub node 101 may be configured to contain an SCM platform for processing data from other nodes (104, 107). In one embodiment, primary node/hub 101 comprises one or more servers 102 operatively coupled to one or more terminals 103. Primary node 101 is communicatively coupled to network 112, which in turn is operatively coupled to supply chain nodes 104, 107. Nodes 104, 107 may be configured as standalone nodes, wherein each node 104, 107 comprises network servers 105, 108 and terminals 106, 109, respectively.

Nodes 104, 107 may be configured as assembly nodes, developer nodes, part nodes, programming nodes, supplier nodes, manufacturer nodes and/or any other suitable supply chain nodes for a single aspect, i.e., hardware or software or firmware or data management, for a particular product or service, such as health care. Each of the illustrated nodes may be configured to collect, store, and process relevant supply chain-related data and transmit the SCM data to primary node 101 via network 112. Primary node 101 may further be communicatively coupled to one or more data services 110, 111 which may be associated with governmental, monetary, economic, meteorological, etc., data services. Services 110, 111 may be third-party services configured to provide general environmental data relating to a particular supply chain, such as interest rate data, tax/tariff data, weather data, trade data, currency exchange data, and the like, to further assist in SCM processing. Primary node/hub 101 may be “spread” across multiple nodes, rather than comprising a single node, may access data at any one or more of a plurality of layers from nodes 104, 107, and may be capable of applying a selectable one or more algorithms, applications, calculations, or reporting in relation to any one or more data layers from nodes 104, 107. In short, separate supply chains, such as that shown in FIG. 1, would then typically exist for other aspects of a product or service in addition to the one supply chain shown.

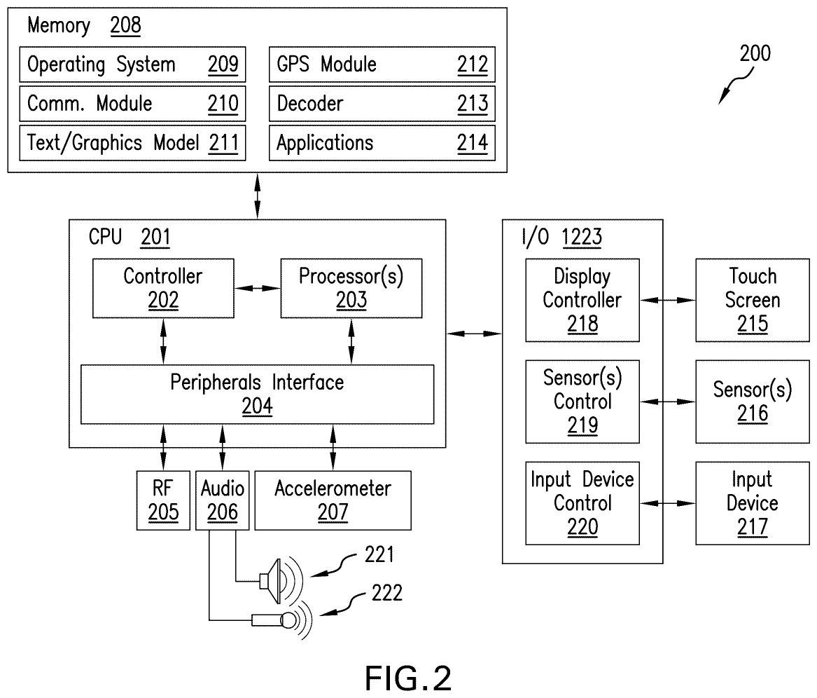

FIG. 2 is an exemplary embodiment of a computing device 200 which may function as a computer terminal (e.g., 103) to perform the disclosed computing operations, and may be a desktop computer, laptop, tablet computer, smart phone, or the like. Actual devices may include greater or fewer components and/or modules than those explicitly depicted in FIG. 2. Device 200 may include a central processing unit (CPU) 201 (which may include one or more computer readable storage mediums), a memory controller 202, one or more processors 203, a peripherals interface 1204, RF circuitry 205, audio circuitry 206, a speaker 221, a microphone 222, and an input/output (I/O) subsystem 223 having display controller 218, control circuitry for one or more sensors 216 and input device control 214. These components may communicate over one or more communication buses or signal lines in device 200. It should be appreciated that device 200 is only one example of a multifunction device 200, and that device 200 may have more or fewer components than shown, may combine two or more components, or a may have a different configuration or arrangement of the components. The various components shown in FIG. 2 may be implemented in hardware or a combination of hardware and tangibly-embodied, non-transitory software, including one or more signal processing and/or application specific integrated circuits.

Data communication with device 200 may occur via a direct wired link or data communication through wireless, such as RF, interface 205, or through any other data interface allowing for the receipt of data in digital form. Decoder 213 is capable of providing data decoding or transcoding capabilities for received media, and may also be enabled to provide encoding capabilities as well, depending on the needs of the designer. Memory 208 may also include high-speed random access memory (RAM) and may also include non-volatile memory, such as one or more magnetic disk storage devices, flash memory devices, or other non-volatile solid-state memory devices. Access to memory 208 by other components of the device 200, such as processor 203, decoder 213 and peripherals interface 204, may be controlled by the memory controller 202. Peripherals interface 204 couples the input and output peripherals of the device to the processor 203 and memory 208. The one or more processors 203 run or execute various software programs and/or sets of instructions stored in memory 208 to perform various functions for the device 200 and to process data including SCM data. In some embodiments, the peripherals interface 204, processor(s) 203, decoder 213 and memory controller 202 may be implemented on a single chip, such as a chip 201. In some other embodiments, they may be implemented on separate chips.

The RF (radio frequency) circuitry 205 receives and sends RF signals, also known as electromagnetic signals. The RF circuitry 205 converts electrical signals to/from electromagnetic signals and communicates with communications networks and other communications devices via the electromagnetic signals. The RF circuitry 205 may include well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 205 may communicate with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), BLE, Bluetooth, Wireless Fidelity (Wi-Fi) (e.g., IEEE 802.11a, IEEE 802.11b, IEEE 802.11g and/or IEEE 802.11n), voice over Internet Protocol (VOIP), Wi-MAX, a protocol for email (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), and/or Instant Messaging and Presence Service (IMPS)), and/or Short Message Service (SMS)), or any other suitable communication protocol, including communication protocols not yet developed as of the filing date of this document.

Audio circuitry 206, speaker 221, and microphone 222 may provide an audio interface between a user and the device 200. Audio circuitry 1206 may receive audio data from the peripherals interface 204, converts the audio data to an electrical signal, and transmits the electrical signal to speaker 221. The speaker 221 converts the electrical signal to human-audible sound waves. Audio circuitry 206 also receives electrical signals converted by the microphone 221 from sound waves, which may include audio. The audio circuitry 206 converts the electrical signal to audio data and transmits the audio data to the peripherals interface 204 for processing. Audio data may be retrieved from and/or transmitted to memory 208 and/or the RF circuitry 205 by peripherals interface 204. In some embodiments, audio circuitry 206 also includes a headset jack for providing an interface between the audio circuitry 206 and removable audio input/output peripherals, such as output-only headphones or a headset with both output (e.g., a headphone for one or both ears) and input (e.g., a microphone).

I/O subsystem 223 couples input/output peripherals on the device 200, such as touch screen 215 and other input/control devices 217, to the peripherals interface 204. The I/O subsystem 223 may include a display controller 218 and one or more input controllers 220 for other input or control devices. The one or more input controllers 220 receive/send electrical signals from/to other input or control devices 217. The other input/control devices 217 may include physical buttons (e.g., push buttons, rocker buttons, etc.), dials, slider switches, joysticks, click wheels, and so forth. In some alternate embodiments, input controller(s) 220 may be coupled to any (or none) of the following: a keyboard, infrared port, USB port, and a pointer device such as a mouse, an up/down button for volume control of the speaker 221 and/or the microphone 222. Touch screen 215 may also be used to implement virtual or soft buttons and one or more soft keyboards.

Touch screen 215 provides an input interface and an output interface between the device and a user. The display controller 218 receives and/or sends electrical signals from/to the touch screen 215. Touch screen 215 displays visual output to the user. The visual output may include graphics, text, icons, video, and any combination thereof (collectively termed “graphics”). In some embodiments, some or all of the visual output may correspond to user-interface objects. Touch screen 215 has a touch-sensitive surface, sensor or set of sensors that accepts input from the user based on haptic and/or tactile contact. Touch screen 215 and display controller 218 (along with any associated modules and/or sets of instructions in memory 208) detect contact (and any movement or breaking of the contact) on the touch screen 215 and converts the detected contact into interaction with user-interface objects (e.g., one or more soft keys, icons, web pages or images) that are displayed on the touch screen. In an exemplary embodiment, a point of contact between a touch screen 215 and the user corresponds to a finger of the user. Touch screen 215 may use LCD (liquid crystal display) technology, or LPD (light emitting polymer display) technology, although other display technologies may be used in other embodiments. Touch screen 215 and display controller 218 may detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with a touch screen 215.

Device 200 may also include one or more sensors 216 such as optical sensors that comprise charge-coupled device (CCD) or complementary metal-oxide semiconductor (CMOS) phototransistors. The optical sensor may capture still images or video, where the sensor is operated in conjunction with touch screen display 215. Device 200 may also include one or more accelerometers 207, which may be operatively coupled to peripherals interface 1204. Alternately, the accelerometer 207 may be coupled to an input controller 214 in the I/O subsystem 211. The accelerometer is preferably configured to output accelerometer data in the x, y, and z axes.

In one embodiment, the software components stored in memory 208 may include an operating system 209, a communication module 210, a text/graphics module 211, a Global Positioning System (GPS) module 212, audio decoder 1213 and applications 214. Operating system 209 (e.g., Darwin, RTXC, LINUX, UNIX, OS X, Windows, or an embedded operating system such as VxWorks) includes various software components and/or drivers for controlling and managing general system tasks (e.g., memory management, storage device control, power management, etc.) and facilitates communication between various hardware and software components. A SCM processing platform may be integrated as part of operating system 209, or all or some of the disclosed portions of SCM processing may occur within the one or more applications 214. Communication module 210 facilitates communication with other devices over one or more external ports and also includes various software components for handling data received by the RF circuitry 205. An external port (e.g., Universal Serial Bus (USB), Firewire, etc.) may be provided and adapted for coupling directly to other devices or indirectly over a network (e.g., the Internet, wireless LAN, etc.).

Text/graphics module 211 includes various known software components for rendering and displaying graphics on a screen and/or touch screen 215, including components for changing the intensity of graphics that are displayed. As used herein, the term “graphics” includes any object that can be displayed to a user, including without limitation text, web pages, icons (such as user-interface objects including soft keys), digital images, videos, animations and the like. Additionally, soft keyboards may be provided for entering text in various applications requiring text input. GPS module 212 determines the location of the device and provides this information for use in various applications. Applications 214 may include various modules, including address books/contact list, email, instant messaging, video conferencing, media player, widgets, instant messaging, camera/image management, and the like. Examples of other applications include word processing applications, JAVA-enabled applications, encryption, digital rights management, voice recognition, and voice replication. Under one embodiment, a 3D object may have access to any or all of features in memory 208.

The embodiments solve the disadvantages of the known art discussed above, namely the discretization of various supply chain aspects of product and service development and manufacturing, through the creation of a digital device twin using a digital device engine resident on a digital device platform. That is, supply chain development in the embodiments includes a digitization of the supply chain for the hardware of a device, including all materials lists, design revisions, manufacturing data, manufacturing machines, algorithms, and locations, and so on; in conjunction with a digital model of all firmware that operates the hardware of the device; all software that runs on the device; all software that remotely communicates with the device; all communication methodologies over which this communication occurs; and all data developed regarding operation of the hardware, firmware, and purpose-driven software aspects of the device during operation, and so on.

Thereby, the digital device engine provides a holistic digital profile of the end-to-end development, manufacturing, and operation of the device, including lifetime horizons for all hardware features of the device, firmware, and software of the device, as well as all algorithms that are device-purpose based, and all data generated therefrom. Accordingly, the embodiments provide a new and unified supply chain that is truly end-to-end and holistic for all aspects of a product or service, which allows for risk assessment, lifetime prediction, obsoletion prediction, connectivity and performance characteristics, and so on. The embodiments combine all of the foregoing in a digital model that can be readily subjected to all relevant guidelines, rules, and laws, notwithstanding that the guidelines, rules and laws were each previously applied only to discrete aspects of the product or service, namely via independent application to the hardware, firmware, software, algorithms, data storage, data use, and user data and device information.

Of course, it also goes without saying that the embodiments can provide the aforementioned features for existing devices, i.e., devices that are already completely designed, are already being manufactured, and/or that are already providing the purpose-driven services. That is, the embodiments may also aid in the assessment of the health of existing devices, their connectivity, their data generation, their data management, their sustainability, their manufacturing and supply chain optimization and risk, and their operability within all relevant rules, guidelines, laws and requirements. In such an embodiment for an existing product or service, it may be necessary that a digital model, or at least particular aspects and features thereof, of the holistic product or service be uploaded to the disclosed digital device engine.

The skilled artisan will appreciate, in light of the discussion herein, that whether the holistic digital device model is developed using the disclosed engine or is uploaded to the disclosed engine, the disclosed digital device engine may eventually provide a vast knowledge store against which comparisons can occur via application of machine learning. That is, the disclosed engine may perform machine learning by developing a “knowledge” of features/costs/designs/manufacturing techniques that work best regarding bills of materials, particular parts, hardware devices, firmware, software, connectivity, data storage and management, and so on, from other devices similarly provided in like-verticals, or from/for other devices that include similar hardware, firmware, or software components in like- or unlike-verticals.

It goes without saying that such data regarding other devices may be maintained discretely by the disclosed digital platform based a confidential electronic profile uniquely for each customer's product or service, and consequently, data may be anonymously applied for the machine learning discussed throughout in such a manner that each customer receives solely suggestions, feedback, modifications, and/or recommendations, without knowledge of where such information comes from or what such information relates to. Thus, the disclosed engine allows for continuous innovation, troubleshooting, problem-solving, and improved data use and management for any of a variety of devices that previously would have required discrete supply chains for each aspect thereof in the prior art, as referenced above. Further, the embodiments thus provide not only improvements in the supply chain for hardware, firmware, software, and data storage and management, but additionally provide improved generation of device data, improved device and software performance, improved manufacturing, improved manufacturing costs and efficiency, decreased risk, and which allow for the building of improved device software and algorithms to better generate desired output data.

Additionally, the above variety of improved outcomes and device lifetimes, and decreased risk may be particularly impactful in fields in which device operation and data generation is high stress and high impact, such as in the medical field. Moreover, use of the digital device engine for feedback and/or design may additionally be highly impactful in heavily regulated areas for data generation, such as in the health care, automotive, aerospace, utilities, or precious materials industries, by way of non-limiting example.

The features discussed throughout are provided by a holistic digital device engine operating on a digital device platform. The disclosed engine may provide the requisite correlations, design capabilities, and other digital mirror features discussed throughout. The disclosed engine may operate on the digital device platform, which may additionally provide the requisite features, input and output connectivity to the engine, connectivity between the engine and third party public or private data stores, data management and storage, and information and data discretization and confidentiality.

The digital device engine may treat each product or service as a module-by-module architecture, in which each hardware, firmware and software aspect of the architecture is modeled in the digital device engine. That is, the device itself may serve as a hardware model module, including the hardware performance, the operation context, the operation environment, the hardware connectivity, and the data generated. The engine may include a firmware and/or software model module, which may include firmware operation to service the device, the underlying capabilities provided to the device, as well as connectivity options, thin or thick client operability, processing and storage capabilities, and so on. The engine may include a data management and flow module, which may include the actual software algorithms that provide the device hardware and firmware with their intended purpose, the data generated in accordance with the device outcome, the competency of the data generated, the specific outcomes and the manner of data management regarding the outcomes, and the compliance with specifications of the device purpose. The engine may also provide a supply chain management module to manage the previously discrete supply chains of all of the foregoing, which may include manufacturing methods, software development methods, firmware development methods, risk management, manufacturing risk, bills of materials, device elements and modules, comparative performance and success of data with similar devices, other devices or verticals with the same or similar elements and/or operational goals, and so on.

Thereby, the embodiments can provide a seamless end-to-end supply chain management for a given device. By way of particular example, this may allow for improved hardware battery performance in a certain device, such as by the use of battery sensor data and comparison to battery performance of similar devices using different batteries or different battery connectivity hardware. Likewise, this may allow for communication management for the device's communication hardware, such as by comparison to different communication methods and parts for similar devices, as may use other communication networks, such as GSM v. LTE. Yet further, the disclosed engine may allow for correlation between device usage, such as high-risk device usage for medical patients, together with operational risks and overall effectiveness, such as using any of a variety of communication types. Finally, the engine can offer not only a projected success of an end-to-end design, but design suggestions for improved success in any given context, such as weather monitoring, health care such as heart monitoring, automobile or airplane component devices, and so on.

Yet further, substantial time and expense is saved using the disclosed engine over the known art, particularly for product development and product improvement. For example, development of previously discrete aspects of the supply chain in a single, uniform digital format, along with a anonymized comparison to other similar devices, and in conjunction with simulated and actual operation and outcome data, all without purchasing any components or services for prototyping or experimentation, allows for tremendous expediency in end-to-end product and service design over the known art, as well as highly minimized cost in developing a product or service as compared to the known art.

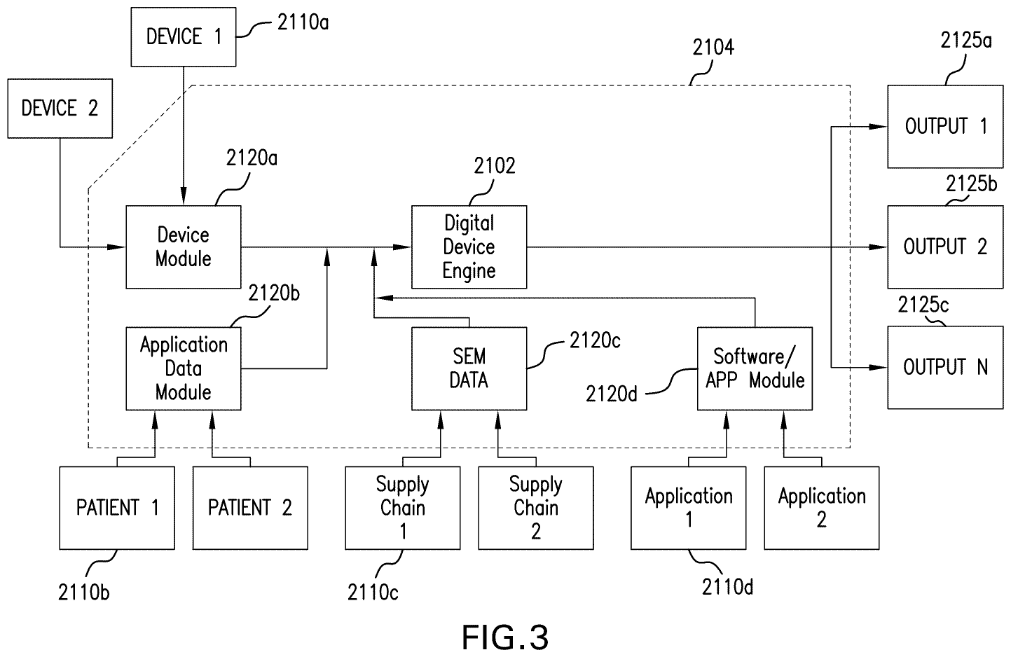

FIG. 3 illustrates the disclosed digital health engine 2102 operating on the digital health platform 2104. As shown, the platform 2104 receives a variety of inputs 2110a, b, c . . . to various modules 2120a, b, c . . . , including but not limited to: an active device module 2120a that receives data from multiple devices, such as data that may include connectivity, performance, operating environment, operating context, and so on for currently operating devices; an application data input module 2120b, which may receive incoming data from multiple points of product or service operation that relates to the core purpose of the product or service, such as application data that may include patient data, user data, operational data, risk data, success rate data, ultimate outcome data, performance v. specification data, and the like; a supply chain management input module 2120c, which may additionally include the digital device mirroring discussed throughout, and which may include supply chain related data related to the design, manufacturing and operation of both the software and hardware, and additionally to the firmware, of an offered product or service and its manufacturing methods and specifications, such as may include comparative manufacturing data related to similar devices, parts and materials listings, supply chain risk factors, optional manufacturing methods or parts, comparative data to other devices with similar contextual goals, comparative data to other devices that include similar parts and materials, modular manufacturing, and the like; and a software/firmware input module 2120d, at which is received knowledge of information related to applications, application context, device part drivers, external third party linked information, and the like related to the core operation of the product or service, as well as peripheral operations, such as communications, administrative access, updates, downloads and uploads, and the like; and finally, the digital health engine 2102 that is resident on the platform 2104 that provides optimization in light of the received inputs 2110a, b, c . . . to/modules of the platform 2104, and that provides correlation for the end-to-end optimization of the holistic device, wherein the correlation may include data weighting, specification comparisons, comparative data (such as anonymously) to other devices, outcome goals, and the like. Each of the foregoing may be provided from the platform 2104 as output 2125a, b, c . . . .

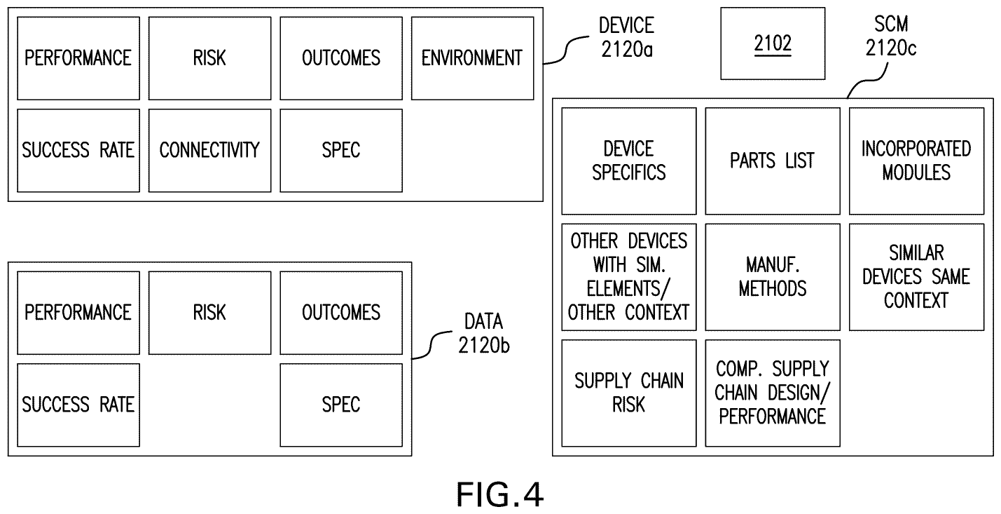

FIG. 4 illustrates several unique exemplary modules 2120a, 2120b, 2120c . . . associated with an exemplary digital health engine 2102 that may reside on the digital health platform 2104 in the unique context of a health-care patient. As illustrated, the digital health platform may include the digital engine and a plurality of inter-related modules for a given product or service, such as in relation to the device data, the performance, risk, outcomes, operating environment, success rate, connectivity, and comparison to specifications, for each device from which data is input to the digital health platform. Relatedly, the digital engine may include an optimization module for the received supply chain management data, which may include device specifics, parts lists, incorporated modules, other devices with similar elements, and in similar contexts, manufacturing methods, supply chain risk, and comparative supply chain designs with optimized or inadequate performance. Yet further and as illustrated, the application data module for the digital health engine may include data manipulations unique to the contextual application for each product or service. Thus, in the illustration, the patient data for the disclosed product and service is assessed as to performance of the device on the patient, the risk to the patient and in the use of the device, the patient outcomes for patients subjected to the device, the success rate of the use of the device, the comparison to specifications for the goal in patient treatment for the device, and the applicable rules, laws, and guidelines with regard to the patient's data as well as with regard to operation of the device.

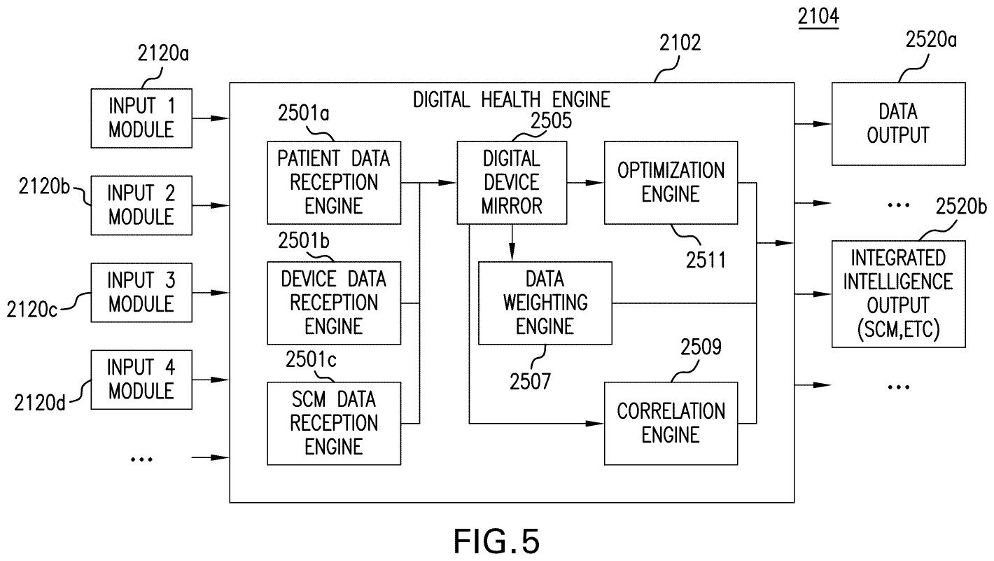

FIG. 5 is a block diagram illustrating with greater specificity the presence of the digital health engine 2102 on the digital health platform 2104. As shown, the digital health platform 2104 may include data reception modules 2102 for a variety of purposes and which ultimately feed the reception engines 2501a, b, c . . . data health engine. The digital health engine may include the disclosed digital mirroring 2505 of the actual product or service, and may additionally include various modules that allow for the assessments discussed herein. Such modules may include, by way of non-limiting example, data weighting 2507, correlation 2509, optimization 2511, and the like. Thereby, the disclosed digital health platform may receive input data from a variety of sources regarding a variety of aspects of a product or service, and may provide, via manipulations from the digital engine, outputs/output data 2520a, b . . . that may comprise modifications to the device, modifications to the service, modification to an application, modification to data processing and storage, supply chain management, operational risk management, and the like.

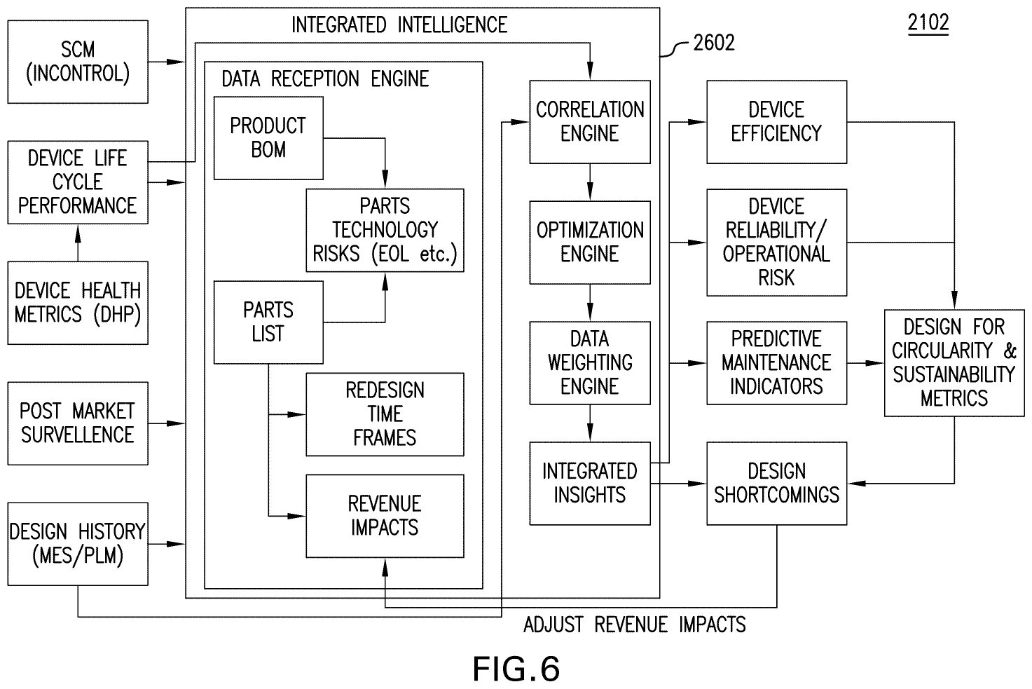

FIG. 6 is a block diagram illustrating with greater particularity an exemplary supply chain intelligence engine feature 2602 within the digital device health engine 2102. As shown, the particular supply chain data may include bills of materials, parts lists, part risks such as obsoletion and end of life, designs and redesigns, and cost versus revenue. This data may be provided upon reception at an input module to the variety of modules discussed herein as forming part of the digital health engine, including but not limited to a correlation module, an optimization module, a data weighting module, and an integration of prior learnings in a learning module.

The learning module may then, in conjunction with the other modules, provide a variety of data that may improve the supply chain for the relevant product or service, for a similar product or service, or for a different product or service that shares similar parts or applications. Such learning module outputs may improve device efficiency, improve device reliability, decrease operational risks, provide predictive lifetime and maintenance indications, provide design shortcomings, and improve the foregoing aspects by providing designs for improved operability, circularity, sustainability, and so on.

A module within the digital health platform may then modify cost and revenue calculations and provide these via a feedback loop to the relevant module. Moreover, such feedback may be received globally from the output of the particular module, and be fed back as an input to the module, such as along with module-unique data related to the purpose of the module. By way of example, a supply chain module may receive data related to supply chain management, device life cycles, parts life cycles, device operational/health metrics, market surveillance, historical designs and design modifications, and the like.

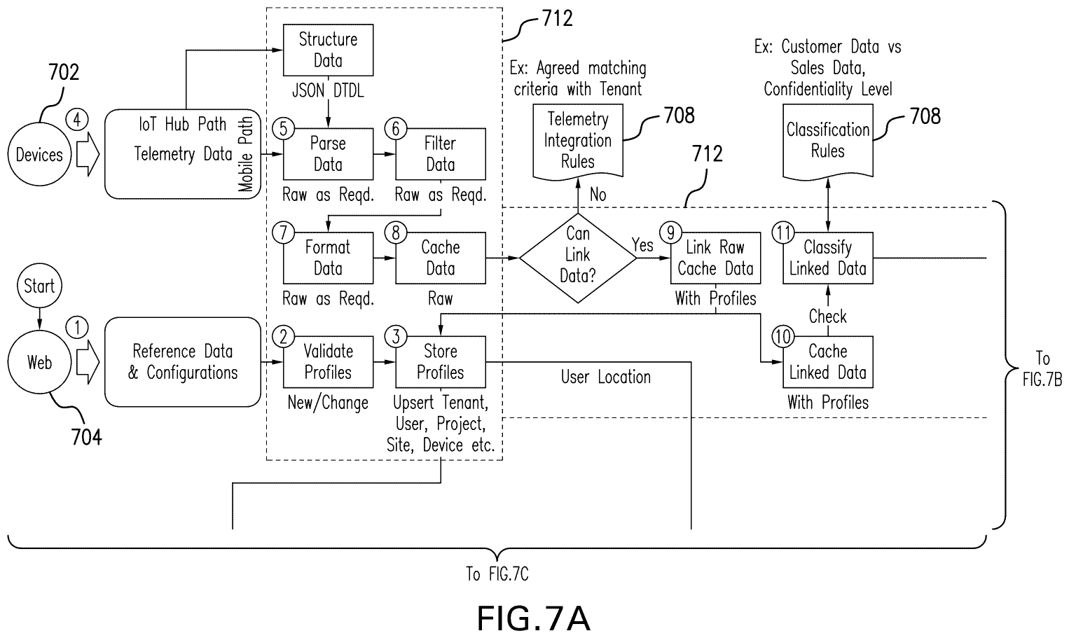

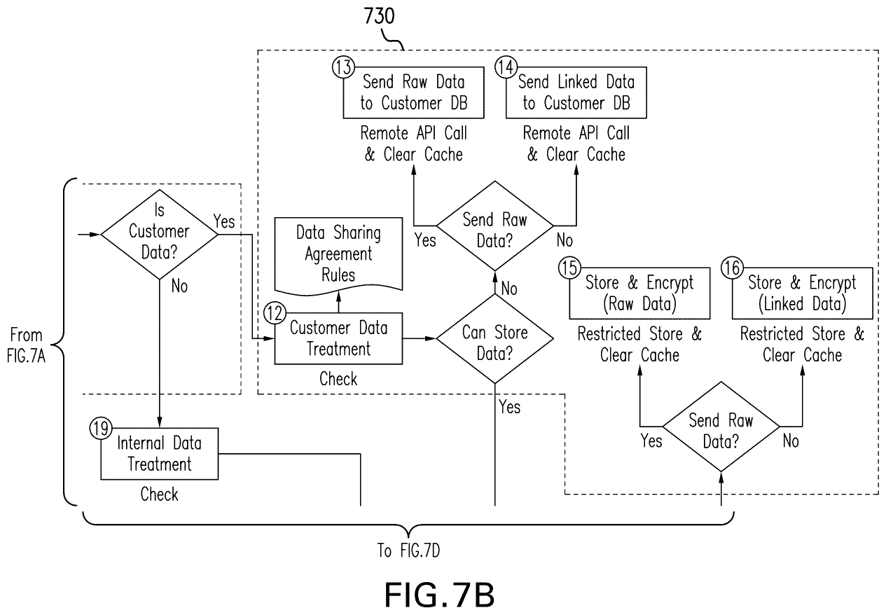

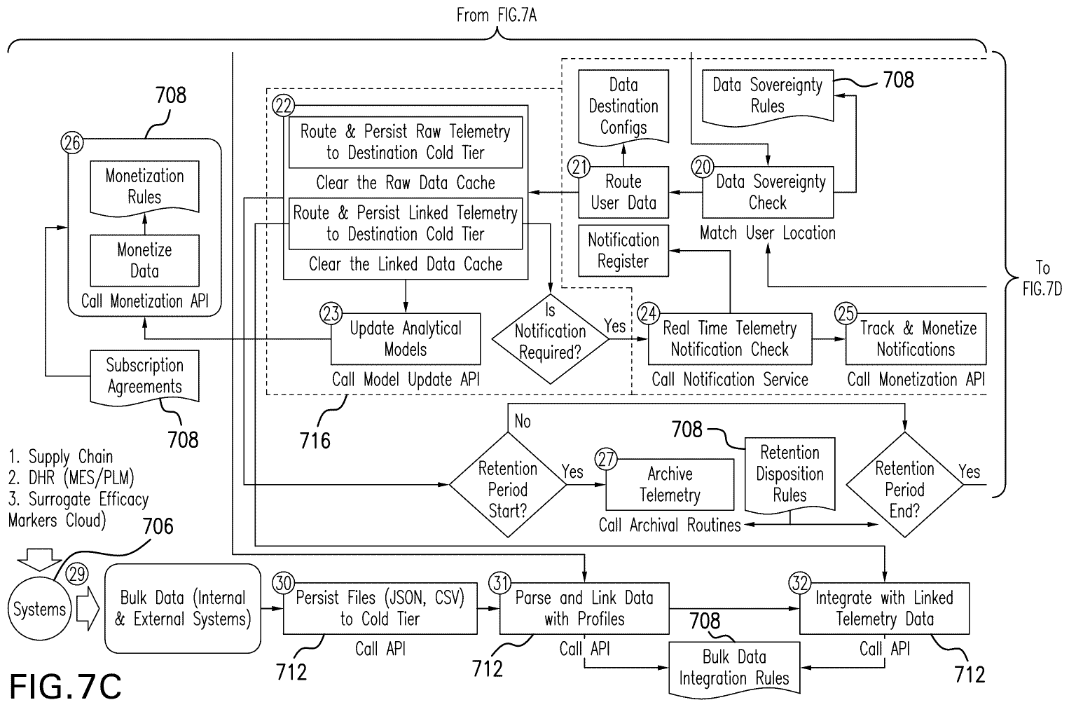

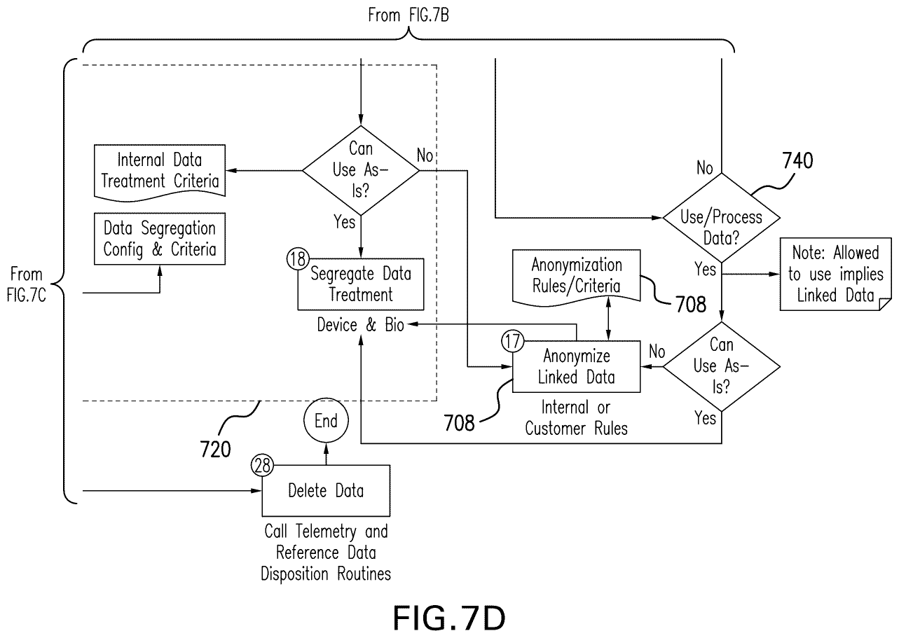

FIG. 7 is a flow diagram of the operation of the digital engine 102 according to an exemplary flow in accordance with the embodiments. As shown, information to allow for the digital engine to operate may be input from a variety of sources, including but not limited to: operating device data 702; reference, configuration, environmental, and other third party data 704 from the Web; bulk data sources 706, such as may relate to the current or comparable supply chains, for example; as well as various rules/laws/guidelines 708, such as may relate to monetization, cost management, confidentiality and/or anonymization, subscription or membership, integration and classification, and the like.

The foregoing types of information may be data-managed, such as in relation to data parsing, formatting, validation, linking, structuring, and storage 712. Data may be manipulated, persisted and updated 716. Data may be subjected to various checks, routing, and rules applications 720. Data may be treated, sent, or stored 730. And, needless to say, data may be processed 740.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

1. A digital device health platform for optimizing an offering of a product, comprising:
a plurality of supply chains, each of which contributes a different aspect of the product;
a plurality of input modules comprising:
at least one third party data input for receiving third party data;
a supply chain management input for receiving supply chain data from each of the plurality of supply chains;
an application data input for receiving data regarding operation of at least one application operating in conjunction with hardware of the product to provide a purpose of the product;
a device hardware data input for receiving data regarding operation of the hardware;
a software application input for receiving purpose data from the at least one application; and
a firmware input for receiving data regarding operation of firmware drivers for the hardware;
a digital device health engine that receives processed data from each of the input modules, and that processes the received processed data, via at least one computer processor processing non-transitory computing code, to generate a holistic digital twin of all aspects of the product corresponding to the plurality of the inputs, which includes the supply chain data from each of the plurality of supply chains, wherein the corresponding comprises at least a correlating to an optimized level of performance of the purpose, a comparing to a plurality of rules governing the performance of the purpose, and feedback from machine learning regarding anonymized prior similar iterations of other products similar to the product as reflected by the third party data; and
a plurality of outputs reflecting the corresponding to a graphical user interface.

2. The digital device health platform of claim 1, wherein the received data regarding operation of the hardware includes at least one of connectivity data, performance data, operating environment data and operating context data for currently operating hardware.

3. The digital device health platform of claim 1, wherein the received receiving data regarding operation of at least one application operating in conjunction with hardware of the product includes at least one of patient data, user data, operational data, risk data, success rate date, ultimate outcome data and specification data.

4. The digital device health platform of claim 1, wherein the digital device health engine includes a digital device mirroring.

5. The digital device health platform of claim 1, wherein the digital device health engine includes an optimization module for the received supply chain management data.

6. The digital device health platform of claim 1, wherein the digital device health engine includes at least one of a correlation engine and a data weighing engine.

7. The digital device health platform of claim 1, wherein the application data input includes data manipulations unique to the contextual application for the product.

8. The digital device health platform of claim 1, wherein the digital device health engine includes a supply chain intelligence engine.
